(19) United States
(12) Patent Application Publication
(10) Pub. No.: US 2012/0208242 A1
(43) Pub. Date: Aug. 16, 2012

(54) Method and RNA Reactor for Exponential Amplification of RNA

(75) Inventor: Jacques Rohayem, Dresden (DE)

(73) Assignee: RiboxX GmbH, Radebeul (DE)

(21) Appl. No.: 13/502,413

(22) PCT Filed: Oct. 21, 2010
(86) PCT No.: PCT/EP10/65912

§ 371 Date: Apr. 17, 2012

(30) Foreign Application Priority Data
Oct. 21, 2009 (EP) 09173699.1
Nov. 10, 2009 (EP) 09175565.2

(51) Int. Cl. C12P 19/34; B01J 19/12; C12M 1/40

(52) U.S. Cl. 435/91.21; 435/289.1; 204/157.68

(57) ABSTRACT

The present invention relates to a method for exponential amplification of RNA using a primer independent RNA-dependent RNA polymerase (RdRp) wherein reactants are premixed cycle and then transferred into the reaction chamber in which the steps of polymerisation of the complementary strand and separation of the resulting double-stranded RNA occur. The invention also relates to a RNA reactor for carrying out the exponential RNA amplification.

The present invention relates to a method for exponential amplification of RNA using a RNA-dependent RNA polymerase (RdRp) wherein reactants are premixed and then transferred into the reaction chamber in which the steps of polymerisation of the complementary strand and separation of the resulting double-stranded RNA occur. The invention also relates to a an RNA reactor for carrying out the exponential RNA amplification.

In comparison to DNA amplification by PCR, existing RNA amplification methods suffer from several drawbacks: protocols for mRNA amplification using T7 polymerase (SMART™ mRNA Amplification Kit User Manual, Clontech Laboratories, Inc., 28 April 2008; U.S. Pat. No. 5,962,271, U.S. Pat. No. 5,962,272) include complex and time consuming enzymatic steps:

1) reverse transcription step of producing a double-stranded cDNA from the RNA which is to be amplified. This occurs usually with a primer-dependent RNA-dependent DNA-polymerase, i.e. from Avian Myeloblastosis Virus (AMV) or Molooney Murine Leukemia Virus (MuLV).

2) The produced double-stranded DNA-Template is then used as a template to synthesisze RNA by the T7 polymerase. The T7-Polymerase is a primer-dependent DNA-dependent RNA-Polymerase and requires a T7 specific promoter sequence within the primer sequence for initiation of polymerisation.

Amplification of RNA by the T7 Polymerase occurs in a linear fashion.

Another enzyme which has been suggested for RNA amplification is Qβ replicase (see WO 02/092774 A2). Qβ replicase is an RNA-dependent RNA-polymerase that needs a primer having a sequence-specific recognition site for initiation of RNA polymerisation. Protocols of this type only achieve linear RNA amplification.

Furthermore, RNA amplification using polymerases from bacteriophages Phi-6 to Phi-14 (cf. WO 01/46396 A1) requires the presence of a specific promoter sequence. Phi-6 to Phi-14 enzymes are RNA-dependent RNA-polymrases. Also in this case only linear amplification has been achieved with such enzymes.

WO 2007/12329 A2 discloses a method for preparing and labelling RNA using a (RNA-dependent RNA-polymerase) RdRp of the family of Caliciviridae. The authors show successful de novo RNA synthesis from single-stranded RNA (ssRNA) templates in the presence or absence of a RNA-synthesis initiating oligonucleotide (oligoprimer with a length less than 10 nt) and also envisage repeated cycling of RNA synthesis and denaturation of the double-stranded RNA (dsRNA) products. Exponential RNA amplification is not shown in WO 2007/12329 A2.

The technical problem underlying the present invention is to provide an efficient system for exponential amplification of RNA.

The solution to the above technical problem is provided by the embodiments of the present invention as characterised in the claims.

In particular, the present invention provides, according to a first aspect, a method for exponential amplification of RNA comprising the steps of:
(a) mixing single-stranded RNA (ssRNA), a primer-independent RNA-dependent RNA polymerase (RdRp), NTPs (i.e. ribonucleotides rATP, rCTP, rGTP and rUTP (rNTPs) and/or modified and/or labelled rNTPs and/or deoxyribonucleotides (dNTPs) and/or modified and/or labelled dNTPs), reaction buffer and, optionally, RNA-synthesis initiating oligonucleotide in a mixing chamber;
(b) transferring the mixture of step (a) into a reaction chamber;
(c) optionally, annealing said RNA-synthesis initiating oligonucleotide to said ssRNA;
(d) incubating said mixture in said reaction chamber under conditions so that the primer-independent RdRp synthesizes a RNA strand complementary to said ssRNA de novo or, optionally, said RdRp elongates said RNA-synthesis initiating oligonucleotide (oiligoprimer) hybridised to said ssRNA to form double-stranded RNA (dsRNA);
(e) separating said dsRNA formed in step (d) into ssRNA strands;
(f) mixing primer-independent RdRp, NTPs, reaction buffer and, optionally, oligoprimer in said mixing chamber;
(g) transferring the mixture of step (f) into said reaction chamber;
(h) repeating steps (d) to (g) or, optionally, (c) to (g) at least 5 times, preferably 5 to 100 times;
(i) performing a final incubation step (d) to form final dsRNA; and, optionally,
(j) recovering said final dsRNA from said reaction chamber.

According to the present invention, step (f) and (g), respectively, may be carried out in each cycling step (h). However, it is also contemplated that “fresh” reactants, in particular RdRp and/or NTPs, may be added (i.e. transferred into the reaction chamber according to step (g) as defined above) after a series, e.g. 2 to 10 cycles of polymerisation and strand separation. Thus, fresh reactants may be added at every 2^{nd} to 10^{th}, preferably at every ₂^{nd}, 3^{rd} or 4^{th} cycle in the present RNA amplification protocol. It is clear for the skilled person that, in this embodiment, the time point of mixing fresh reactants may be chosen freely within the time window of carrying out the cycles of polymerisation and strand separation.

It is preferred that the RdRp has a “right hand conformation” and that the amino acid sequence of said protein comprises a conserved arrangement of the following sequence motifs:

| | |
|---|---|
| a. | XXDYS |
| b. | GXPSG |
| c. | YGDD |
| d. | XXYGL |
| e. | XXXXFLXRXX |

with the following meanings:
D: aspartate
Y: tyrosine
S: serine
G: glycine
P: proline
L: leucine
F: phenylalanine
R: arginine
X: any amino acid.

The so-called “right hand conformation” as used herein means that the tertiary structure (conformation) of the protein folds like a right hand with finger, palm and thumb, as observed in most template-dependent polymerases.

The sequence motif “XXDYS” is the so-called A-motif. The A-motif is responsible for the discrimination between ribonucleosides and deoxyribonucleosides. The motif “GXPSG” is the so-called B-motif. The B-motif is conserved within all representatives of this RdRP family of the corresponding polymerases from the Caliciviridae. The motif “YGDD” (“C-motif”) represents the active site of the enzyme. This motif, in particular the first aspartate residue (in bold, YGDD) plays an important role in the coordination of the metal ions during the Mg²⁺/Mn²⁺-dependent catalysis. The motif “XXYGL” is the so-called D-motif. The D-motif is a feature of template-dependent polymerases. Finally, the “XXXXFLXRXX” motif (E-motif) is a feature of RNA-dependent RNA polymerases which discriminates them from DNA-dependent RNA polymerases.

Typical representatives of the above types of RdRps are the corresponding enzymes of the calicivirus family (Caliciviridae). The RdRps of the calicivirus family are capable of synthesizing complementary strands using as a template any ssRNA template in vitro, including heterologous viral, eukaryotic and prokaryotic templates. The ssRNA template may be positive stranded or negative stranded.

The above-defined RdRp is capable of synthesizing a complementary strand both by elongation of a RNA-synthesis initiating oligonucleotide and by de novo synthesis in the absence of a RNA-synthesis initiating oligonucleotide. The RNA-synthesis initiating oligonucleotide, if desired, may be a sequence specific RNA-synthesis initiating oligonucleotide or may be a random RNA-synthesis initiating oligonucleotide or may be an oligo-T-RNA-synthesis initiating oligonucleotide. More details of the characteristic features of the calicivirus RdRp and of RNA-syntesis initiating oligonucleotides (oligoprimer) can be found in WO 2007/012329 A2.

According to the present invention, the terms “primer”, “oligoprimer” and “RNA-synthesis initiating oligonucleotide” are used interchangeably and refer to a short single-stranded RNA or DNA oligonucleotide (e.g. 5 to 10 nucleotides in length, typically for amplifying shorter RNA templates; longer oligoprimers (e.g. having a length of 10 to 20 or more nucleotides) may be used for amplifying larger RNA species) capable of hybridizing to a target ssRNA molecule under hybridization conditions such that the RdRp is able to elongate said primer or RNA-synthesis oligonucleotide, respectively, under RNA polymerization conditions. In contrast to other RNA-dependent RNA polymerases, e.g. RNA-dependent RNA polymerases such as replicases of the Qβ type, the RNA polymerases of the caliciviruses do not require primers having a specific recognition sequence for the polymerase to start RNA synthesis. Thus, a “primer”, oligoprimer” or “RNA-synthesis initiating oligonucleotide” as used herein is typically a primer not having such recognition sequences, in particular, of RNA polymerases. Furthermore, the calicivirus RNA polymerases are different from usual DNA-dependent RNA polymerases such as T7 RNA polymerase in that they do not require specific promoter sequences to be present in the template.

Preferably, the RNA-dependent RNA-polymerase is an RdRp of a human and/or non-human pathogenic calicivirus. Especially preferred is an RdRp of a norovirus, sapovirus, vesivirus or lagovirus, for example the RdRp of the norovirus strain HuCV/NL/Dresden174/1997/GE (GenBank Acc. No. AY741811) or of the sapovirus strain pJG-Sap01 (GenBank Acc. No. AY694184) or an RNA-dependent RNA polymerase of the vesivirus strain FCV/Dresden/2006/GE (GenBank Acc. No. DQ424892).

According to especially preferred embodiments of the invention the RdRp is a protein having an amino acid sequence according SEQ ID NO: 1 (norovirus-RdRp), SEQ ID NO: 2 (sapovirus-RdRp) or SEQ ID NO: 3 (vesivirus-RdRp). The person skilled in the art is readily capable of preparing such RdRp, for example by recombinant expression using suitable expression vectors and host organisms (cf. WO 2007/012329 A2). To facilitate purification of the RdRp in recombinant expression, it is preferred that the RdRp is expressed with a suitable “tag” (for example GST or (His)₆-tag) at the N- or C-terminus of the corresponding sequence. For example, a histidine tag allows the purification of the protein by affinity chromatography over a nickel or cobalt column in a known fashion. Examples of embodiments of RdRPs fused to a histidine tag are the proteins having an amino acid sequence according to SEQ ID NO: 4, SEQ ID NO: 5, SEQ ID NO: 6 or SEQ ID NO: 7. SEQ ID NO: 4 corresponds to a norovirus-RdRp having a histidine tag. SEQ ID NO: 5 and SEQ ID NO: 6 correspond to the amino acid sequence of a sapovirus-RdRp having a histidine tag (SEQ ID NO: 5: C-terminal His-tag; SEQ ID NO: 6: N-terminal His-tag). SEQ ID NO: 7 corresponds to the amino acid sequence of vesirius-RdRp having a histidine tag.

SEQ ID NO: 1:
MGGDSKGTYCGAPILGPGSAPKLSTKTKFWRSSTTPLPPGTYEPAYL
GGKDPRVKGGPSLQQVMRDQLKPFTEPRGKPPKPSVLEAAKKTIINV
LEQTIDPPKKWSFTQACASLDKTTSSGHPHHMRKNDCWNGESFTGKL
ADQASKANLMFEGGKNMTPVYTGALKDELVKTDKIYGKIKKRLLWGS
DLATMIRCARAFGGLMDELKAHCVTLPIRVGMNMNEDGPIIFERHSR
YKYHYDADYSRWDSTQQRAVLAAALEIMVKFSSEPHLAQVVAEDLLS
PSVVDVGDFKISINEGLPSGVPCTWQWNSIAHWLLTLCALSEVTNLS
PDIIQANSLFSFYGDDEIVSTDIKLDPEKLTAKLKEYGLKPTRPDXT
EGPLVISEDLNGLTFLRRTVTRDPAGWFGKLEQSSILRQMYWTRGPN
HEDPSETMIPHSQRPIQLMSLLGEAALHGPAFYSKISKLVIAELKEG
GMDFYVPHQEPMFRWMRFSDLSTWEGDRNLAPSFVNEDGVEVDKLAA
ALE
SEQ ID NO: 2:
MKDEFQWKGLPVVKSGLDVGGMPTGTRYHRSPAWPEEQPGETHAPAP
FGAGDKRYTFSQTEMLVNGLKPYTEPTAGVPPQLLSRAVTHVRSYIE
TIIGTHRSPVLTYHQACELLERTTSCGPPVQGLKGDYWDEEQQQYTG
VLANHLEQAWDKANKGIAPRNAYKLALKDELRPIEKNKAGKRRLLWG
CDAATTLIATAAFKAVATRLQVVTPMTPVAVGINMDSVQMQVMNDSL
KGGVLYCLDYSKWDSTQNPAVTAASLAILERFAEPHPIVSCAIEALS
SPAEGYVNDIKFVTRGGLPSGMPFTSVVNSINHMIYVAAAILQAYES
HNVPYTGNVFQVETVHTYGDDCMYSVCPATASIPHAVLANLTSYGLK
PTAADKSDAIKPTNTPVFLKRTFTQTPHGVRALLDITSITRQFYWLK
ANRTSDPSSPPAFDRQARSAQLRNALAYASQNGPVVFDTVRQIAIKT
AQGEGLVLVNTNYDQALATYNAWFIGGTVPDPVGHTTEGTHKIVFEM
E
SEQ ID NO: 3:
MKVTTQKYDVTKPDISYKGLICKQLDEIRVEFKGTRLHVSPAHTDDY
DECSHQPASLGSGDPRCPKSLTAIVVDSLKPYCEKTDGPPHDILHRV
QRMLIDHLSGFVPMNISSEPSMLAAFHKLNHDTSCGPYLGGRKKDHM
IGGEPDKPLLDLLSSKWKLATQGIGLPHEYTIGLKDELRPVEKVQEG
KRRMIWGCDVGVATVCAAAFKGVSDAITANHQYGPVQVGINMDGPSV
EALYQRIRSAAKVFAVDYSKWDSTQSPRVSAASIDILRYFSDRSPIV
DSAANTLKSPPIAIFNGVAVKVTSGLPSGMPLTSVINSLNHCLYVGC
AILQSLRSRNIPVTWNLFSTYDMMTYGDDGVYMFRMMFASVSDQIFA
NLTAYGLKPTRVDKSVGAIEPIDPESVVFLKRTITRTPHGIRGLLDR
GSIIRQFYYIKGENSDDWKTPPKTIDPTSRGQQLWNACLYASQHGPE
FYNKVYRLAEKAVEYEELHFEPPSYHSALEHYNNQFNGVDTRSDQID
ASVMTDLHCDVFEVLE
SEQ ID NO: 4:
MGGDSKGTYCGAPILGPGSAPKLSTKTKFWRSSTTPLPPGTYEPAYL
GGKDPRVKGGPSLQQVMRDQLKPFTEPRGKPPKPSVLEAAKKTIINV
LEQTIDPPEKWSFTQACASLDKTTSSGHPHHMRKNDCWNGESFTGKL
ADQASKANLMFEGGKNMTPVYTGALKDELVKTDKIYGKIKKRLLWGS
DLATMIRCARAFGGLMDELKAHCVTLPIRVGMNMNEDGPIIFERHSR
YKYHYDADYSRWDSTQQRAVLAAALEIMVKFSSEPHLAQVVAEDLLS
PSVVDVGDFKISINEGLPSGVPCTSQWNSTAHWLLTLCALSEVTNLS
PDIIQANSLFSFYGDDEIVSTDIKLDPEKLTAKLKEYGLKPTRPDKT
EGPLVISEDLNGLTFLRRTVTRDPAGWFGKLEQSSILRQMYWTRGPN
EEDPSETMIPHSQRPIQLMSLLGEAALHGPAFYSKISKLVIAELKEG
GMDFYVFRQEPMFRWMRFSDLSTWEGDRNLAPSFVNEDGVEVDKLAA
ALEHHHHHH
SEQ ID NO: 5:
MKDEFQWKGLPVVKSGLDVGGMPTGTRYHRSPAWPEEQPGETHAPAP
FGAGDKRYTFSQTEMLVNGLKPYTEPTAGVPPQLLSRAVTHVRSYIE
TIIGTHRSPVLTYHQACELLERTTSCGPFVQGLKGDYWDEEQQQYTG
VLANHLEQAWDKANKGIAPRNAYKLALKDELRPIEKNKAGKRFLLWG
CDAATTLIATAAFKAVATRLQVVTPMTPVAVGINMDSVQMQVMNDSL
KGGVLYCLDYSKWDSTQNPAVTAASLAILERFAEPHPIVSCAIEALS
SPAEGYVNDIKFVTRGGLPSGMPFTSVVNSINHMIYVAAAILQAYES
HNVPYTGNVFQVETVHTYGDDCMYSVCPATASIFHAVLANLTSYGLK
PTAADKSDAIKPTNTPVFLKRTFTQTPHGVRALLDITSITRQFYWLK
ANRTSDPSSPPAFDRQARSAQLENALAYASQHGPVVFDTVRQIAIKT
AQGEGLVLVNTNYDQALATYNAWFIGGTVPDFVGHTEGTHKIVFEME
HHHHHH
SEQ ID NO: 6:
MKHHHHHHDEFQWKGLPVVKSGLDVGGMPTGTRYHRSPAWPEEQPGE
THAPAPFGAGDKRYTFSQTEMLVNGLKPYTEPTAGVPPQLLSRAVTH
VRSYIETIIGTHRSPVLTYHQACELLERTTSCGPFVQGLKGDYWDEE
QQQYTGVLANHLEQAWDKANKGIAPRNAYKLALKDELRPIEKNKAGK
RRLLWGCDAATTLIATAAFKAVATRLQVVTPMTPVAVGINMDSVQMQ
VMNDSLKGGVLYCLDYSKWDSTQNPAVTAASLAILERFAEPHPIVSC
AIEALSSPAEGYVNDIKFVTRGGLPSGMPFTSVVNSINHMIYVAAAI
LQAYESHNVPYTGNVFQVETVHTYGDDCMYSVCPATASIFHAVLANL
TSYGLKPTAADKSDAIKPTNTPVFLKRTFTQTPHGVRALLDITSITR
QFYWLKANRTSDPSSPPAFDRQARSAQLENALAYASQHGPVVFDTVR
QIAIKTAQGEGLVLVNTNYDQALATYNAWFIGGTVPDPVGHTEGTHK
IVFEME
SEQ ID NO: 7:
MKVTTQKYDVTKPDISYKGLICKQLDEIRVIPKGTRLHVSPAHTDDY
DECSHQPASLGSGDPRCPKSLTAIVVDSLKPYCEKTDGPPHDILHRV
QRMLIDHLSGFVPMNISSEPSMLAAFHKLNHDTSCGPYLGGRKKDHM
IGGEPDKPLLKLLSSKWKLATQGIGLPHEYTIGLKDELRPVEKVQEG
KRRMIWGCDVGVATVCAAAFKGVSDAITANHQYGPVQVGINMDGPSV
EALYQRIRSAAKVFAVDYSKWDSTQSPRVSAASIDILRYFSDRSPIV
DSAANTLKSPPIAIFNGVAVKVTSGLPSGMPLTSVINSLNHCLYVGC
AILQSLESRNIPVTWNLFSTFDMMTYGDDGVYMFPMMFASVSDQIPA
NLTAYGLKPTRVDKSVGAIEPIDPESVVFLKRTITRTPHGIRGLLDR
GSIIRQFYYIKGENSDDWKTPPKTIDPTSRGQQLWNACLYASQHGPE
FYNKVYRLAEKAVEYEELHFEPPSYHSALEHYNNQFNGVDTRSDQID
ASVMTDLHCDVFEVLEHHHHHH

The method of the present invention is suited to provide amplified RNA of all kinds and lengths. The method is particularly useful for providing short RNA molecules for gene silencing applications, either by antisense technology or RNA interference.

Therefore, the ssRNA template to be used in the method of the present invention has preferably a length of 8 to 45 nucleotides, preferably of 15 to 30 nucleotides, preferably of 21 to 28 nucleotides, more preferably of 21 to 23 nucleotides. RNA molecules of the latter length are particularly useful for siRNA applications.

For de novo initiation of RNA synthesis (i.e. in the absence of a primer) it is preferred that the template contains at least 1, more preferred 1, 2, 3, 4 or 5, in particular 1 to 3 C nucleotides at its 3′ end.

Alternatively, the method of the present invention is also useful to provide longer RNA molecules, i.e. the ssRNA template has more than 30 nucleotides. A preferred embodiment of the inventive method makes use of mRNA templates.

In case of amplifying polyadenylated RNA (in particular mRNA) an RNA-synthesis initiating oligonucleotide (oligo- or polyU primer) is required. Correspondingly, amplification of polyguanylated and polyuridylated RNA requires an oligoC (or polyC) and oligoA (or polyA), respectively, primer. In the case of polycytidylated templates RNA synthesis can either be initiated by using an oligoG (or polyG) primer or it can be initiated de novo (i.e. in the absence of an RNA-synthesis initiating oligonucleotide) using GTP in surplus (preferably, 2×, 3×, 4× or 5× more) over ATP, UTP and CTP, respectively.

The method of the present invention is also useful to provide modified RNA molecules, in particular in the context of siRNA production. Thus, it is envisaged to include labelled and/or modified rNTPs or NTPs (such as 2′-or 3′-deoxy-modified nucleotides) in step(s) (a) and/or (f) as defined above.

Chemically modified RNA products of the method of the present invention preferably have an increased stability as compared to the non-modified dsRNA analogues.

Especially for this purpose, the chemical modification of the at least one modified ribonucleoside triphosphate to be incorporated by the RdRp activity into the complementary strand can have a chemical modification(s) at the ribose, phosphate and/or base moiety. With respect to molecules having an increased stability, especially with respect to RNA degrading enzymes, modifications at the backbone, i.e. the ribose and/or phosphate moieties, are especially preferred.

Preferred examples of ribose-modified ribonucleoside triphosphates are analogues wherein the 2′-OH group is replaced by a group selected from H, OR, R, halo, SH, SR, NH₂, NHR,

NR₂ or CN with R being C₁-C₆ alkyl, alkenyl or alkynyl and halo being F, CI, Br or I. It is clear in the context of the present invention, that the term “modified ribonucleoside triphosphate” or “modified ribonucleotide” also includes 2′- or 3′-deoxy derivatives which may at several instances also be termed “deoxynucleotides”.

Typical examples of such ribonucleotide analogues with a modified ribose at the 2′ position include 2′-O-methyl-cytidine-5′-triphosphate, 2′-amino-2′-deoxy-uridine, 2′-azido-2′-deoxy-uridine-5′-triphosphate, 2′-fluoro-2′-deoxy-guanosine-5′-triphosphate and 2′-O-methyl-5-methyl-uridine-5′-triphosphate. For further details with regard to providing chemically modified RNA species by using the method of the present invention it is referred to co-pending International Patent Application No. PCT/EP2009/057119 (published as WO-A-2009/150156).

The method of the present invention is highly flexible with regard to the scale (amount of reactants, reaction volume etc.). For example, the method of the present invention can be carried out in pl to ml scales, e.g. 25 μl to 6 ml, but can upscaled to industrial volumes of, e.g up to 5000 liters.

It is preferred that the mixing volume in step (f) is doubling at each or after a series of cycles (e.g. after 2, 3 or 4 cycles, if step (f) is carried at every 2^{nd}, 3^{rd} or 4^{th} cycle), e.g. starting at 25 μl and after one cycle, increasing to 50 μl, with subsequent increase in the next cycle to 100 μl, and so on and so forth. The reaction volume in the reaction chamber increases (preferably doubles in volume) after each cycle or after a series if cycles of transferring reactants or buffer from mixing to reaction chamber, polymerisation and strand separation by the volume present in the mixing chamber after mixing the reactants.

It is further preferred that the reactants in step(s) (a) and/or (f) are cooled, preferably at a temperature of 2 to 8° C., more preferably at 4° C.

The polymerisation step (d) is generally carried out at temperature of from 28 to 42° C., preferably at 30° C. The polymerisation step (c) is generally carried out for about 15 to about 120 min, more preferred from about 30 min to about 60 min, particularly preferred for 90 min.

The polymerisation step may be carried under shaking at 50 to 600 round per minute, preferably 100 to 400 rounds per minute, most preferably 300 rounds per minute.

The strand separation step (e) may be carried by heat, chemically or enzymatically. If carried out enzymatically, the strand separation step (e) is preferably carried out by an enzyme having strand displacement and/or double-strand unwinding and/or double-strand separation activity.

In case the separation step (e) is embodied as a heat denaturation step, the temperature is generally dependent on the melting temperature of the dsRNA product which is in turn dependent on the length and GC content. As a rule, the heat denaturation is carried out at temperatures of from about 65° C. to about 98° C., more preferred between 75° C. to 95° C. For small interfering RNA species, in particular having a length of 15 to 25 nt, a heat denaturation at about 85° C. may be sufficient.

The separation (e.g. denaturation) step (d) is generally carried out for about 5 min to about 90 min, more preferred from about 15 min to about 30 min, particularly preferred for 60 min.

According to preferred embodiments of the present invention, microwave radiation may be used for carrying out the incubation steps (step (d) and/or (i) and/or the separation step (e). Thus, the reaction composition present in the respective step(s) of the method according to the present invention is exposed to an amount of microwave radiation effective and sufficient to reach and maintain the respective reaction conditions as defined herein.

The term “effective amount of microwave energy” is the amount of microwave energy required for the RNA polymerisation of a complementary strand on a single-stranded polynucleotide template using a primer-independent RdRp as defined in steps (a) and (i) and/or to separate the double-stranded product in step (e). The concrete amount of microwave energy for a given template may be determined by the skilled person using routine experimentation and depends particularly on the length and type of template. For the polymerisation steps (step (d) and/or (i)), the microwave energy may be lower compared to the conditions required in the separation step (b). As used herein the terms “microwave energy”, “microwave (ir)radiation” or “irradiation with microwaves” or simply “microwaves” are used synonymously and relate to the part of the electromagnetic spectrum comprising wavelengths of about 0.3 to 30 cm, corresponding to a frequency of 1 to 100 gigahertz, which is found between the radio and the infra-red regions of the electromagnetic spectrum.

The amount of electromagnetic energy absorbed by a living organism is determined by the dielectric properties of the tissues, cells, and biological molecules.

The generation of the microwave energy for the purposes of the present invention is not critical and can be by any means known to the art. For example, suitable means for applying microwave radiation to reaction compositions according to the invention are microwave ovens into which the reaction chamber may be inserted. Such microwave ovens typically have maximum power levels of from about 500 W to about 1000 W. Even the smallest ovens provide ample levels of microwave irradiation for use in this invention and accordingly, it will be convenient to use lower power settings on ovens in which the output power is adjustable.

Thus, according to preferred embodiments of the inventive methods disclosed herein, the composition is irradiated with microwaves having a frequency of from about 1500 MHz to about 3500 MHz and having a power of from about 50 to about 1000 W.

According to other embodiments of this invention, lower power settings are also used to time-distribute the applied power over a longer time interval and minimize the potential for localized energy uptake and resulting molecular damage. In an especially preferred embodiment, microwave power is applied to the sample over a series of intervals, with “rest” intervals, in which microwave power is not applied to the sample. Power application intervals and rest intervals will usually range from 1 to 60 seconds each, with power application intervals of from 15 to 60 seconds and rest intervals from 0.5 to 5 seconds being preferred. Most preferably, power will be applied for intervals of about 45 seconds, separated by rest intervals of 1 to 2 seconds.

However, especially depending on the length of the single-stranded polynucleotide template, the irradiation step may be carried out in a single application (interval) of microwave energy of a time period of 1 s to 5 min, more preferably 3 s to 120 s. The latter short time periods are especially useful when templates of shorter length (such as templates for preparing short dsRNAs such as siRNAs) are employed.

Further subject matter of the present invention relates to an RNA reactor for large-scale synthesis of RNA comprising:
a mixing chamber having means for mixing reactants, e.g. in a volume of 25 μl to 5000 litres (in the latter case it is designed for industrial, large scale applications), more preferred from 250 μl to 500 ml and means for cooling the mixture of reactants.
a reaction chamber having means for heating and/or for applying microwave radiation to the reaction mixture and having a reaction volume capable of being doubled after having received reactants from the mixing chamber;
a conduct for connecting said mixing chamber with said reaction chamber;
a first storage chamber having cooling means and being connected via a conduct to said mixing chamber;
second and third storage chambers each having cooling means and being connected to said mixing chamber via a common conduct;
pumping means for transferring reactants from said first, second and third storage chambers to said mixing chamber and for transferring reaction mixtures from said mixing chamber to said reaction chamber
   wherein the mixing volume of the mixing chamber is capable of being doubled after having received reactants from said first, second and third storage chambers.

Usually, the reaction chamber is equipped with means for pH and/or temperature measurement, and with means for collecting samples from the reaction mixture present in the reaction chamber. The reaction volume in the reaction chamber may be designed for μl to ml volumes. However, the reaction volume in the reaction chamber may also be designed for industrial, large scale applications with volumes of up to 5000 or 10000 litres. The reaction chamber preferably is equipped with means for shaking the reaction mixture present in the reaction chamber, preferably having a shaking capacity of 50 to 600 rounds per minute, more preferably 100 to 400 rounds per minute, most preferably 300 rounds per minute.

The means for applying microwave irradiation to the reaction mixture comprise a source of microwave radiation. Corresponding devices are known to the person skilled in the art. It is to be noted that the microwave radiation may also be used to heat the reaction mixture to a desired temperature, besides the fact that microwaves as such (i.e. independent of a possible temperature effect on the reaction mixture) accelerate and/or induce the reactions occurring in the polymerisation and/or separation steps. In this respect, the means for applying microwave radiation to the reaction mixture may also be regarded as means for heating the reaction chamber.

According to a preferred embodiment, the first storage chamber is equipped with cooling means for cooling the storage chamber to −20° C. and below. The second and/or third chamber(s) preferably has/have cooling means for cooling the respective storage chamber(s) to temperatures of from 2 to 8° C., more preferably 4° C. Thus the first storage chamber is designed to store RdRp. The second and third storage chambers are used to store NTPs (as defined above), buffer and, optionally, RNA-synthesis initiating oligonucleotide (one of the second and third storage chamber) and ssRNA template (the other of the second and third storage chamber). If present, it is also possible to provide a fourth storage chamber for storing RNA-synthesis initiating oligonucleotide(s) only.

Since the RNA reactor of the present invention is provided for carrying out RNA amplification reactions, it is highly preferred that all components are made RNase free before carrying out RNA amplification reactions. The same holds true for all reactants and liquids used in the method of the present invention.

Furthermore, the reaction chamber preferably has heating means for heating the chamber to a temperature of from 28° C. to 98° C.

The RNA reactor of the present invention is preferably embodied as a high-throughput device employing microliquid handling equipment. Corresponding system components are commercially available.

The figures show:

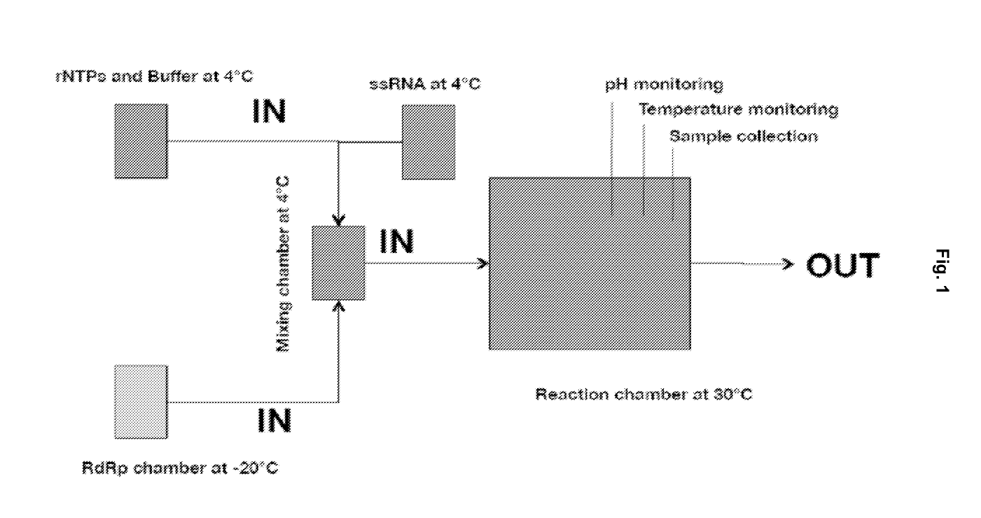

FIG. 1 shows a schematic representation of a preferred embodiment of the RNA reactor according to the present invention.

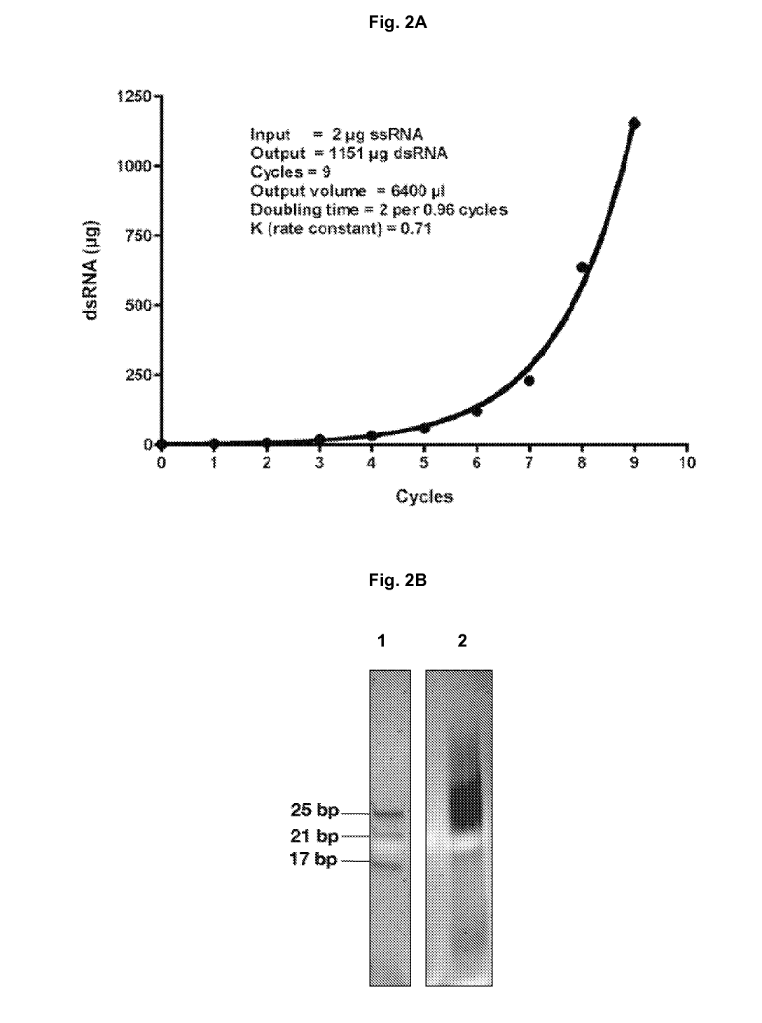

FIG. 2 (A) shows a graphical representation of the amount (pg) of RNA produced by the method of the present invention depending on the number of reaction cycles. (B) shows a photograph of a native 20% polyacylamide gel separation of RNA Marker (corresponding to dsRNA of 17 bp, 21 by and 25 bp; lane 1) and dsRNA product (lane 2) resulting from 9 cycles of a method according to the invention. The amount of dsRNA was determined using the RiboGreen fluorescent dye (Invitrogen) measured on the TECAN Infinite 200.

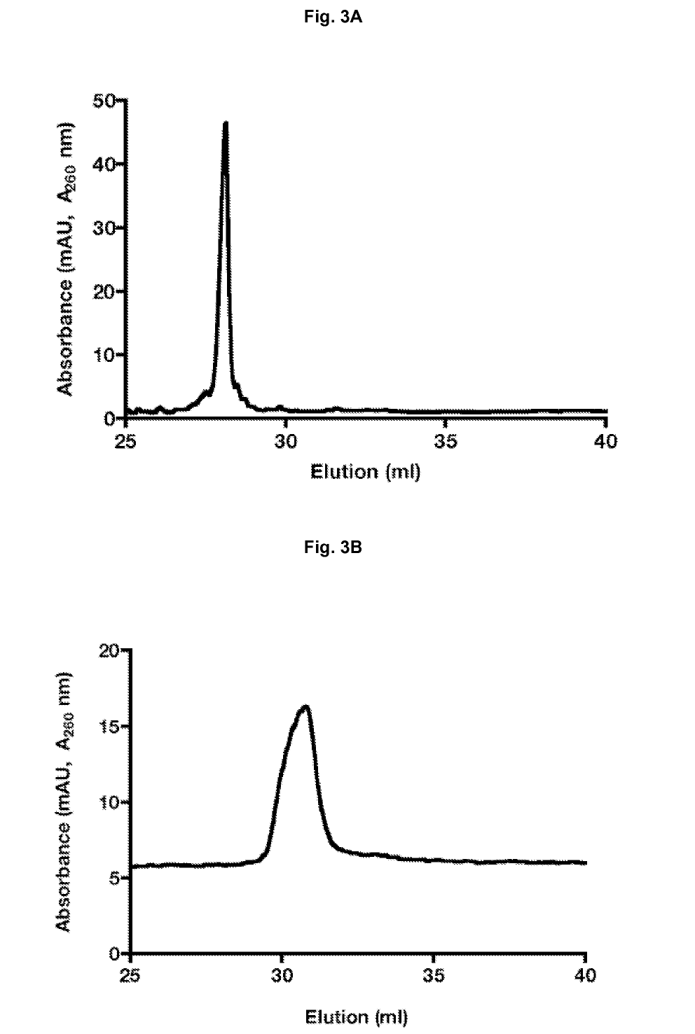

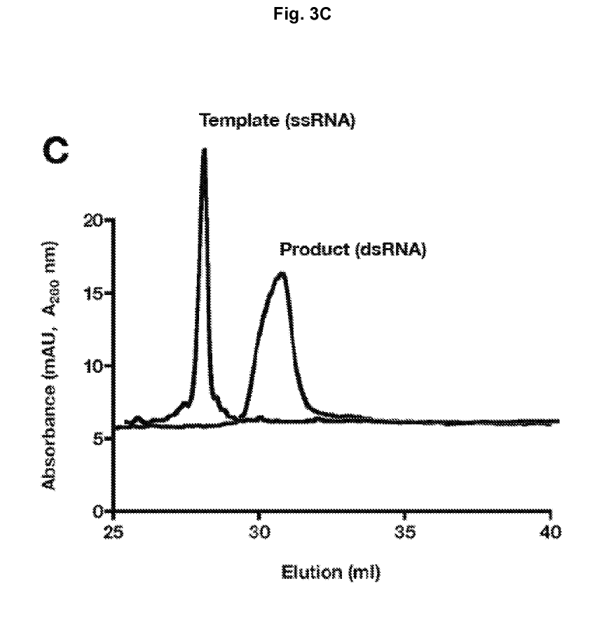

FIG. 3 shows elution profiles of ion exchange chromatographic analyses of ssRNA template (22 nt) (A) and of the dsRNA product resulting from exponential amplification according to the present invention employing the ssRNA template. (C) shows the superposition of (A) and (B).

With reference to FIG. 1, a preferred RNA reactor according to the present invention is characterised as follows:

The RNA reactor has a first storage chamber cooled to −20° C. or below for providing RdRp. Further two storage chambers are present for providing NTPs, buffer and, optionally, RNA-synthesis initiating oligonucleotide (second storage chamber) and ssRNA (third storage chamber), both kept at 4° C. by a cooling mechanism. The reactants (RdRp, NTPS/buffer and ssRNA template) are transferred to the mixing chamber which has cooling means for cooling the mixing chamber to 4° C. The first, second and storage chambers are connected to the mixing chamber via conducts, preferably being cooled to the same temperature as the respective storage chamber. The conduct connecting the second and third storage chambers with the mixing chamber is embodied such that part of said conduct is formed as a common line. The reactants transferred into the mixing chamber are mixed (e.g. by shaking such as at 300 rounds per minute) and then transferred via a conduct into the reaction chamber. The reaction chamber is heated to the appropriate temperature for optimal activity of the RdRp (e.g. 30° C.). The reaction chamber may also (or instead of heating the reaction chamber) be equipped with a source of microwave radiation so as to apply an effective amount of microwave energy to the reaction mixture for polymerisation and/or strand separation steps. The polymerisation temperature may be hold for an appropriate period of time (e.g. 1 to 2 h such as 1.5 h). Especially in case of enhancing polymerisation steps by applying microwave radiation, polymerisation times may be substantially shorter, e.g. down to minutes or even seconds, depending particularly on the type and length of the template as well as on the reaction volume. Polymerisation preferably occurs under shaking conditions, e.g. at 300 rounds per minute. The reaction mixture in the reaction chamber is then heated for denaturation of the dsRNA (e.g. 65 to 96° C., 30 min to 1.5 h). Next, further aliquots of RdRp (from first storage chamber) and of buffer/NTPs (from second storage chamber) are transferred into the mixing chamber (but no ssRNA!), mixed and then transferred into the reaction chamber. The transfer may occur after each cycle, or after a series of cycles (e.g. 3-10 cycles) of polymerisation and strand separation. Further steps of mixing the reactants, transferring into the reaction chamber, polymerisation and denaturation follow as before. This cycling is repeated until the desired amount of product RNA is reached. The temperature and pH conditions in the reaction chamber are monitored via corresponding measuring means. Samples of the reaction mixture can be collected after every or selected cycle(s) of polymerisation and denaturation via known sampling devices. The (final) dsRNA product is collected from the reaction chamber via a conduct.

The present invention is further illustrated by the following non-limiting examples.

### EXAMPLES

### Example 1

### Exponential RNA Amplification Protocol

Using the RNA reactor of FIG. 1, an amplification of the following protocol was performed:

The reaction starts by mixing the template (ssRNA) with RdRp, buffer, and rNTPS in the mixing chamber. The reaction is then transferred to the synthesis chamber (=reaction chamber), where the synthesis of the double-stranded RNA takes place. The procedure is the following: 1) transfer of template, RdRp, rNTPs, buffer into the mixing chamber, 2) mixing of the reaction at 4° C. by shaking (300 rpm for 30 sec.), 3) transfer of the reaction to the synthesis chamber, 4) 1. cycle: 30° C./ 1.5 h, shaking at 300 rpm, 95° C./1 h, 5) transfer of RdRp, rNTPs, Buffer in the mixing chamber (not the ssRNA template!!), 6) mixing of the reaction at 4° C. by shaking (300 rpm for 30 sec.), 7) transfer of the reaction to the synthesis chamber, 8) 2. cycle: 30° C./ 1.5 h shaking at 300 rpm, 95° C./1 h, 9) transfer of RdRp, rNTPs, Buffer in the mixing chamber (not the ssRNA template!!), 10) and so on and so forth. At the end of the cycles, the dsRNA is collected from the synthesis chamber (OUT).

RNA synthesis was performed on a single-stranded RNA template using the RNA-dependent RNA polymerase (RdRp) of a Sapovirus (SEQ ID NO: 2).

The initial reaction mix consisted of 2 pg of the template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. The amplification reaction was performed in 9 successive cycles, each cycle consisting of heating of 30° C. for 90 min, shaking at 300 rpm, followed by denaturation at 95° C. for 60 minutes. After each cycle, an aliquot of the reaction was sampled and the amount of double-stranded RNA determined by using the RiboGreen fluorescent dye (Invitrogen) measured on the TECAN Infinite 200, yielding 1151 μg of double-stranded RNA after 9 cycles. At the beginning of each cycle, RdRp, rNTPS and buffer were added to the reaction at the same concentration as initially described. The total volume of the reaction doubled after each cycle. As shown in FIG. 2A, the amount of product RNA is growing exponentially. Thus, starting from an input of 2 μg ssRNA a yield of 1151 μg of product dsRNA was obtained after 9 cycles (575.5 fold amplification).

Having in mind the drawbacks of prior art RNA amplification methods (see the prior art mentioned above), it is remarkable that the RNA amplification reaction according to the present invention is highly efficient even as compared to established PCR protocols: whereas PCR protocols typically result in 1 to 5 μg after 40 cycles, the RNA amplification protocol of the present invention results in more than 1 mg (!) of dsRNA product after only 9 cycles.

### Example 2

### Analysis of Product dsRNA

The double-stranded RNA product obtained according to Example 1 was visualized on a native 20% polyacrylamide gel by electrophoresis (FIG. 2B). A dsRNA product (lane 2) migrating between the 21 by and 25 by RNA marker (lane 1) is visible.

The double-stranded RNA synthesized as outlined in Example 1 was analysed by ion exchange chromatography using a DNAPak PA100 (Dionex) column. The elution profiles of the ssRNA and synthesized double-stranded RNA are shown in FIG. 3A and B, respectively, superposition of the profiles is shown in FIG. 3C. As can be seen from the panels in FIG. 3, the ssRNA template can be clearly differentiated from the product dsRNA. Thus, the dsRNA product can be successfully prepared by ion exchange chromatography from the reaction mixture.

What is claimed is:

1. A method for exponential amplification of RNA comprising the steps of:
(a) mixing single-stranded RNA (ssRNA), a primer-independent RNA-dependent RNA polymerase (RdRp), NTPs, reaction buffer and, optionally, RNA-synthesis initiating oligonucleotide in a mixing chamber;
(b) transferring the mixture of step (a) into a reaction chamber;
(c) optionally, annealing said RNA-synthesis initiating oligonucleotide to said ssRNA;
(d) incubating said mixture in said reaction chamber under conditions so that the primer-independent RdRp synthesizes a RNA strand complementary to said ssRNA de novo or, optionally, said RdRp elongates said RNA-synthesis initiating oligonucleotide hybridised to said ssRNA to form double-stranded RNA (dsRNA);
(e) separating said dsRNA formed in step (d) into ssRNA strands;
(f) mixing primer-independent RdRp, NTPs, reaction buffer and, optionally, RNA-synthesis initiating oligonucleotide in said mixing chamber;
(g) transferring the mixture of step (e) into said reaction chamber;
(h) repeating steps (d) to (g) or, optionally, (c) to (g) at least 5 times;
(i) performing a final incubation step (d) to form final dsRNA; and, optionally, (j) recovering said final dsRNA from said reaction chamber.

2. The method of claim 1 wherein steps (d) to (g) or, optionally, (c) to (g) are repeated 5 to 100 times in step (h).

3. The method of claim 1 or 2 wherein the primer-independent RdRp has a “right hand conformation” and the amino acid sequence of said RdRp comprises a conserved arrangement of the following sequence motifs:
a. XXDYS
b. GXPSG
c. YGDD
d. XXYGL
e. XXXXFLXRXX
with the following meanings:
D: aspartate
Y: tyrosine
S: serine
G: glycine
P: proline
L: leucine
F: phenylalanine
R: arginine
X: any amino acid.

4. The method of claim 3 wherein the primer-independent RdRp is an RdRp of the Caliciviridae family.

5. The method of claim 4 wherein the primer-independent RdRp is an RdRp of a noroviurs, sapovirus, vesivirus or lagovirus.

6. The method of claim 5 wherein the primer-independent RdRp is selected from the group consisting of an RdRp of the norovirus strain HuCV/NL/Dresden174/1997/GE (GenBank Acc. No. AY741811), an RdRp of the sapovirus strain pJG-Sap01 (GenBank Acc. No. AY694184), and an RdRp of the vesivirus strain FCVfDresden/2006/GE (GenBank Acc. No. DQ424892).

7. The method of claim 6 wherein the primer-independent RdRp has an amino acid sequence selected from the group consisting of SEQ ID NO: 1, SEQ ID NO: 2, SEQ ID NO: 3, SEQ ID NO: 4, SEQ ID NO 5:, SEQ ED NO: 6 and SEQ ID NO: 7.

8. The method of claim 1 wherein the ssRNA template has a length of from 15 to 30, preferably 21 to 28 nucleotides, more preferably 21 to 23 nucleotides.

9. The method of claim 1 wherein the ssRNA template has a length of more than 30 nucleotides.

10. The method of claim 9 wherein the ssRNA template is mRNA.

11. The method of claim 1 wherein the reaction volume in steps (d) and (f) is doubled in each cycle of step (h).

12. The method of claim 1 wherein steps (f) and (g) are carried out at every 2^{nd} to 10^{th} cycle of step (h).

13. The method of claim 12 wherein the reaction volume in steps (d) and (f) is doubled in each cycle of step (h) in which said steps (f) and (g) are carried out.

14. The method of claim 1 wherein step(s) (a) and/or (0 is/are carried out at a temperature of from 2 to 8° C., preferably at 4° C.

15. The method of claim 1 wherein step (d) is carried out at a temperature of from 28 to 37° C., preferably 30° C.

16. The method of claim 1 wherein step (d) is carried out under shaking.

17. The method of claim 16 wherein the shaking is carried out at 50 to 600 rounds per minute, preferably 100 to 400 rounds per minute, most preferably 300 rounds per minute.

18. The method of claim 1 wherein step (e) is carried by heat denaturation, chemically or enzymatically.

19. The method of claim 18 wherein the enzymatical separation of the dsRNA strands is carried out by a double-strand unwinding activity.

20. The method of claim 18 wherein the heat denaturation is carried at a temperature of from 65° C. to 98° C.

21. The method of claim 1 wherein the steps (d) and/or (e) and/or (i) are carried out under microwave irradiation.

22. An RNA reactor for large-scale synthesis of RNA comprising
a mixing chamber having means for mixing reactants;
a reaction chamber having means for heating and/or applying microwave radiation to the reaction mixture and having a reaction volume capable of being doubled after having received reactants from the mixing chamber;
a conduct for connecting said mixing chamber with said reaction chamber;
a first storage chamber having cooling means and being connected via a conduct to said mixing chamber;
second and third storage chambers each having cooling means and being connected to said mixing chamber via a common conduct;
pumping means for transferring reactants from said first, second and third storage chambers to said mixing chamber and for transferring reaction mixtures from said mixing chamber to said reaction chamber
wherein the mixing chamber has a mixing volume capable of being doubled after having received reactants from said first, second and and third storage chambers.

23. The RNA reactor of claim 21 wherein the reaction chamber has means for measuring pH and/or temperature.

24. The RNA reactor of claim 21 wherein the reaction chamber has means for collecting samples from the reaction mixture present in said reaction chamber.

25. The RNA reactor of claim 21 wherein the first storage chamber has cooling means for cooling said storage chamber to −20° C. and below.

26. The RNA reactor of claim 21 wherein the second and third storage chamber and the mixing chamber have cooling means for cooling said chambers to 2 to 8° C., preferably at 4° C.

27. The RNA reactor of claim 21 wherein the reaction chamber has heating means for heating said chamber to a temperature of from 28 to 98° C.

28. The RNA reactor of claim 21 wherein the reaction chamber has means for shaking the reaction mixture present in said reaction chamber.


SEQUENCE LISTING (7 sequences)

SEQ ID NO: 1 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 2 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 3 - protein, 533 aa, Vesivirus
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLE

SEQ ID NO: 4 - protein, 526 aa, Artificial
  Norovirus-RdRp having C-terminal his tag
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 5 - protein, 526 aa, Artificial; identical to SEQ ID NO: 4
  Sapovirus-RdRp having C-terminal His-tag

SEQ ID NO: 6 - protein, 523 aa, Artificial
  Sapovirus-RdRp having N-terminal His-tag
MKHHHHHHDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 7 - protein, 539 aa, Artificial
  Vesivirus-RdRp having C-terminal His-tag
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLEHHHHHH